(19) United States
(12) Patent Application Publication
(10) Pub. No.: US 2006/0191535 A1
(43) Pub. Date: Aug. 31, 2006

(54) Treatment of decompression sickness with inhaled nitric oxide gas

(75) Inventors: Thomas H. Shaffer, Chadds Ford, PA (US); Joseph R. Tuckosh, Hockessin, DE (US); Thomas L. Miller, Wilmington, DE (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Nemours Foundation, Wilmington, DE (US)

(21) Appl. No.: 11/065,748

(22) Filed: Feb. 25, 2005

(51) Int. Cl. A61M 16/00; A62B 7/00; F16K 31/02

(52) U.S. Cl. 128/204.18; 128/204.22

(57) ABSTRACT

The present invention includes a method for treating an individual suffering from decompression sickness, a gas mixture that can be used to treat the individual, and an apparatus that can be used to administer the gas mixture. The method includes administering a gas mixture of oxygen and a therapeutically effective amount of nitric oxide gas to the individual. The gas mixture can include a mixture of oxygen, helium and, nitric oxide gases. The gas mixture can be administered using an apparatus that can be worn by the individual. The apparatus includes dispensers for gases, a gas blender to mix the gases, an inspiratory passage, a face mask substantially conformable with the individual's face, and an expiratory passage. When the individual wearing the face mask inhales, the gas mixture travels from the gas blender, through the inspiratory passage to the face mask. The gas mixture is then inhaled by the individual. When the individual exhales a breath, the breath travels from the face mask through the expiratory passage.

### TECHNICAL FIELD

The invention relates to a method, composition and apparatus that can be used for treating decompression sickness.

### BACKGROUND OF THE INVENTION

Decompression sickness (“DCS”) is a condition that results from the dissolution of gas bubbles (usually nitrogen) into tissues of an individual. The dissolution is generally caused when the individual is exposed to a relatively rapid decrease in environmental pressure.

Broken down by symptomatology, DCS generally falls into one of five categories: (1) limb bends, (2) cerebral bends, (3) spinal cord bends, (4) inner ear bends, and (5) lung bends. Limb bends occurs when gas deposition in tissues causes a poorly-localized “pain-only” syndrome. The most common area of localized pain is in the shoulder. Limb bends can be an indicator of a more serious case of DCS. Cerebral bends presents itself with stroke-like symptoms due to paradoxical arterial gas embolism, de novo arterial gas formation, and/or cerebral edema. The spinal cord bends results from transverse paresis caused by retrograde venous thrombosis with patchy necrosis and edema of the spinal cord. There is a predilection with spinal cord bends for damage to high lumbar nerve roots due to lack of collateral circulation in the area. The inner ear bends results from the development of bubble formation and hemorrhage in labyrinthine fluid spaces and vasculature. The lung bends occurs when excessive venous bubbles develop and release vasoactive substances causing pulmonary irritation and bronchoconstriction. The primary symptoms are substernal chest pain, dyspnea, and cough.

DCS can be caused by a variety of factors, but most common are: rapid ascent from a deep scuba dive (generally depths greater than about 10 meters or about 33 feet); rapid ascent in an airplane with an unpressurized cabin; rapid loss of pressure in an airplane (e.g., loss of cabin pressure at high altitudes); sub aqueous tunnel work (e.g., caisson work); inadequate pressurization/denitrogenation when flying; and flying to a high altitude too soon after scuba diving.

Of these factors, the most common cause of DCS occurs from scuba divers ascending too quickly from a relatively deep dive. During deep dives, divers are exposed to higher and higher ambient pressures as they descend. Because of the higher pressures, the inert gases such as nitrogen and helium, which are included in the breathing gases of the diver, are adsorbed into the tissues of the body in higher concentrations than normal. When a diver ascends from the dive, the ambient pressure is reduced causing the absorbed gases to come back out of solution and form “micro bubbles” in the blood. If the ascent is done slowly, the micro bubbles will safely leave the body through the lungs, i.e., expiration. However, during a rapid ascent not all of the micro bubbles leave the body, thereby resulting in DCS.

To prevent DCS with deep scuba dives, decompression schedules have been formulated. These schedules establish a protocol for ascent with depths and time at those depths that a diver should follow as he/she ascends. To some extent these decompression schedules are experimental and thus are not a guarantee against DCS. Further, there are times when the decompression schedules are not followed, either inadvertently (e.g., miscalculation) or intentionally (e.g., getting a diver to the surface for immediate medical treatment for a wound or other physical ailment). Thus, a need for a treatment for DCS still exists.

Currently, the primary treatment for DCS is hyperbaric oxygen (“HBO”) therapy. HBO therapy is a mode of therapy in which the patient breathes 100% oxygen at pressures greater than normal atmospheric pressure. Generally, hyperbaric oxygen therapy involves the systemic delivery of oxygen at levels 2-3 times greater than atmospheric pressure. The oxygen under pressure reduces the micro bubble size in the patient, creating a pressure gradient for nitrogen gas expulsion and forcing oxygen into ischemic tissue.

HBO therapy is conducted in pressurized chambers. For these chambers to operate effectively, a minimum of 400-500 square feet of space is generally required for a single occupancy chamber. Multiple occupancy chambers can require as much as 10,000 square feet of space. The single occupancy and multiple occupancy chambers each require sophisticated equipment and structural design to generate and accommodate the elevated pressures. Due to the size and sophistication necessary to operate the pressurized chambers, the chambers are not typically located in close proximity to areas where treatment of DCS is most needed (e.g., dive sites). As a result, treatment for DCS by HBO therapy can be delayed for many hours.

HBO therapy is also disadvantageous in that in smaller, single occupancy chambers, the patient is left in relative isolation. This is a special concern with patients suffering from a severe case of DCS or with patients who are suffering from conditions in addition to DCS that require medical personnel to be in close proximity with the patient (e.g., having a wound that requires suturing). The small chambers act as a barrier, preventing the medical personnel from closely monitoring the patient and preventing the medical personnel from administering medical services while the patient is receiving HBO therapy. The small chambers are also a concern with patients who are claustrophobic.

Other treatments for DCS are also known, such as 100% oxygen at atmospheric pressure by mask, dextran and standard replacement fluids to correct hypovolemia, and injectable steroids. These treatments are not fully effective in isolation. Rather, these alternative treatments are adjunctive therapies, i.e., treatments used together with the primary treatment (HBO therapy) to assist the primary therapy.

### SUMMARY OF THE INVENTION

One embodiment of the present invention includes a method for treating an individual with decompression sickness. The method includes administering a gas mixture to lungs of the individual. The gas mixture includes oxygen and a therapeutically effective amount of nitric oxide gas. The gas mixture can be administered before the onset of decompression sickness or after decompression sickness has afflicted the individual.

Another embodiment of the present invention includes a gas mixture including a mixture of oxygen, helium and, nitric oxide gases. The gas mixture can be administered as a therapeutic treatment for decompression sickness, provided the nitric oxide is present in the gas mixture in a therapeutically effective amount.

A further embodiment of the present invention includes an apparatus for administering the gas mixtures of the present invention. The apparatus can be worn by an individual to whom administration of the gas mixtures is desired. The apparatus includes dispensers for gases, a gas blender to mix the gases, an inspiratory passage, a face mask substantially conformable with the individual's face, and an expiratory passage. When the individual wearing the face mask inhales, the gas mixture travels from the gas blender, through the inspiratory passage to the face mask. The gas mixture is then inhaled by the individual. When the individual exhales a breath, the breath travels from the face mask through the expiratory passage. The apparatus can be used in conjunction with the gas mixtures of the present invention in the therapeutic treatment for decompression sickness.

### BRIEF DESCRIPTION OF THE DRAWINGS

For the purpose of illustrating the invention, there is shown in the drawing one embodiment of the invention that is presently disclosed; it being understood, however, that this invention is not limited to the precise arrangements and instrumentalities particularly shown.

FIG. 1 is a profile view of an individual wearing one embodiment of an apparatus that can be used to administer at least one embodiment of a gas mixture of the present invention.

### DETAILED DESCRIPTION OF THE DRAWINGS

One embodiment of the present invention relates to a therapeutic treatment for decompression sickness (“DCS”). As used herein, DCS refers to any condition caused by a relatively rapid decrease in environmental pressure that results from gas micro bubbles, primarily nitrogen, coming out of solution in bodily fluids and tissues. Although the focus of the detailed description below is on DCS, the present invention is not so limited. The present invention can be used to treat dysbarism (e.g., DCS, arterial gas embolism, and barotrauma) or any other similar disorder, the treatment of which involves expiring gases out of the lungs of an individual.

To treat an individual suffering from decompression sickness, one embodiment of the present invention includes administering a gas mixture to the lungs of the individual. The gas mixture comprises oxygen and a therapeutically effective amount of nitric oxide (“NO”) gas. Because, as part of the therapy, the individual can inspire the NO, the treatment is herein referred to as “inspired nitric oxide therapy” or “iNO therapy”.

In this embodiment, the therapeutically effective amount of NO improves lung function in the individual. The improved function increases ventilation in the lungs. The increased ventilation increases the expiration of the micro bubbles caused by DCS, thereby assisting in preventing or eliminating DCS.

A therapeutically effective amount of NO is a concentration of NO in a gas mixture that, when administered to a lung of an individual, is effective in treating and/or preventing DCS. Preferably, the therapeutically effective amount of NO is in concentrations from about 1 part per million (“ppm”) in the gas mixture to about 100 ppm in the gas mixture. More preferably, the therapeutically effective amount is in concentrations from about 5 ppm in the gas mixture to about 80 ppm in the gas mixture. Although these are the preferred concentrations, other concentrations are contemplated to be within the scope of the present invention, understanding that NO delivery in too high of a concentration can be toxic.

NO is a highly reactive free radical compound produced by many cells of the body. NO is naturally formed within the vascular endothelial cells from L-arginine and molecular oxygen in a reaction catalyzed by NO synthase. The endothelium (inner lining) of blood vessels use nitric oxide to signal the surrounding smooth muscle to relax. The relaxation of the muscle dilates the blood vessels, allowing for an increase in blood flow. Thus, NO acts as a natural vasodilator.

The problems with relying on naturally produced NO in treating DCS are that (1) NO is not naturally present in amounts that are effective in adequately treating DCS, and (2) NO generally does not travel great distances in the body. NO is generally consumed close to where it is synthesized. In fact, NO essentially acts in paracrine or even autocrine fashion, effecting only cells near its point of synthesis. Thus, to be effective on a given area of the body, NO must be present in effective amounts in that area of the body. To ensure that the NO is in an effective amount in the area to be treated, the NO is artificially administered from outside the body to the subject area.

In the present invention, the therapeutic treatment of DCS entails delivering NO to the lungs of an individual. Thus, inhaled (or inspired) NO (“iNO”) is administered. When inhaled, the iNO signals the muscle surrounding the blood vessels in the lungs to relax. The relaxation of the muscle dilates the blood vessels, allowing for a substantial increase in pulmonary blood flow in the individual. Thus, the iNO acts as a potent pulmonary vasodilator.

The increased blood flow in turn reduces the ventilation-perfusion (“V/Q”) mismatch, improves the gas exchange in the lungs, and enhances nitrogen washout.

Healthy individuals have a slight V/Q imbalance in the lungs because the distributions of inspired air and pulmonary blood flow in normal individuals are neither uniform nor proportionate to each other. Greater V/Q mismatch is present in the vast majority of individuals suffering from lung diseases. V/Q mismatch in both healthy and diseased individuals can create dead space or non-ventilated areas in the lungs, which are areas where the exchange of oxygen and carbon dioxide with the pulmonary blood does not occur.

A special advantage of iNO as a pulmonary vasodilator and consequently as a means to improve gas exchange, is iNO's selectivity. iNO dilates the pulmonary capillaries, in particular the pulmonary capillaries that are in contact with the ventilated aveoli, while having no effect on the resistance of the systemic vasculature. In contrast, capillaries in communication with the non-ventilated alveoli are constricted due to the low iNO concentration. The result is a blood perfusion redistribution towards the ventilated lung areas.

Because iNO predominately dilates the well-ventilated alveoli, an individual receiving iNO therapy has more blood being directed to the well ventilated areas of the lung. The result is a greater gas exchange in the lung. When the blood being directed contains DCS micro bubbles, the greater gas exchange results in a greater amount of the DCS micro bubbles being safely expired. Thus, iNO therapy is effective in treating DCS.

The iNO therapy of the present invention can be performed as a one time treatment or the therapy can be repeated. The iNO therapy can be performed for a short period or for an extended period. The duration of treatment will depend on several factors including, but not limited to, the severity of the DCS and the physical characteristics of the individual. Although the iNO therapy is contemplated to have an immediate (less than 5 minutes) impact on washout, preferred treatment durations range from about 1 hour to about 1 day. Longer durations may be necessary for more severe cases of DCS.

Although iNO therapy is described as an independent treatment for DCS, it is contemplated that the treatment can be used in combination with other known treatments of DCS. For example, iNO therapy can be used in conjunction with hyberbaric oxygen (“HBO”) therapy. The iNO therapy can be performed as an individual is being transported to a HBO facility. It can be performed while an individual is receiving HBO therapy. It can be performed after an individual receives HBO therapy. It can be performed as any combination of before, during, and/or after the HBO therapy. It is contemplated that the combination of iNO therapy and HBO therapy will reduce the total time necessary for treatment of an individual as compared to the individual being treated with either iNO therapy or HBO therapy alone. Shorter treatment times reduce the amount of time an individual must be present in the HBO facility and reduce the costs associated with operating the HBO facility.

The iNO therapy can also be performed along with the application of continuous positive airway pressure. Continuous positive pressure in conjunction with iNO therapy results in an increase in lung volume recruitment and an increase in pulmonary blood flow, which, in turn, allows for a greater volume of gas exchange. The larger the volume of gas exchange, the greater amount of DCS micro bubbles that can be expired by an individual. Continuous positive airway pressure can be applied by any means in the medical field now known or later developed.

The iNO therapy can also be performed where the gas mixture, in addition to including oxygen and nitric oxide gas, includes helium gas. Helium gas is commonly used in the diving industry as a mixture with oxygen for deep dives. During deep dives, divers are exposed to increased atmospheric pressures. Oxygen toxicity, which includes pulmonary oxygen toxicity and central nervous system toxicity, occurs when a person, usually a scuba diver, is exposed to elevated levels of oxygen for several hours or to high pressure oxygen for extended periods of time (the range of time varies depending on the degree of pressure).

To prevent oxygen toxicity during deep dives, divers inspire a mixture of oxygen and an inert gas. The inert gas dilutes the oxygen, preventing oxygen toxicity from occurring. At relatively shallow depths, the inert gas is generally nitrogen. As the dive depths increase, and the partial pressures of the gas increases, nitrogen begins to have a narcotic effect on the diver. To prevent such an effect in deep dives, helium is used as the inert gas instead of nitrogen. Thus, helium gas is generally available at dive locations where deep dives are anticipated.

Although it is known to administer helium to divers during deep dives, helium has not been used in combination with iNO to treat DCS. Preferably, the helium in the gas mixture of the present invention is from about 50% to about 80% of the gas mixture based on volume. More preferably, the helium in the gas mixture is from about 60% to about 70% of the gas mixture based on volume. Preferably, the oxygen in the gas mixture of the present invention is in concentrations from about 20% to about 40% of the gas mixture based on volume. More preferably, the oxygen in the gas mixture is from about 25% to about 30% of the gas mixture based on volume. The oxygen should not be below 20% of the gas mixture based on volume.

It is contemplated that the iNO therapy can be administered as a prophylactic therapy before an individual shows symptoms of DCS or as a treatment after the individual is afflicted with DCS. The prophylactic therapy could be administered, for example, to a diver who has no outwardly symptoms of DCS, but conditions (e.g., violating dive tables on ascent) indicate that the diver may become afflicted with DCS. Such prophylactic therapy could treat the DCS before the illness reaches an advanced stage.

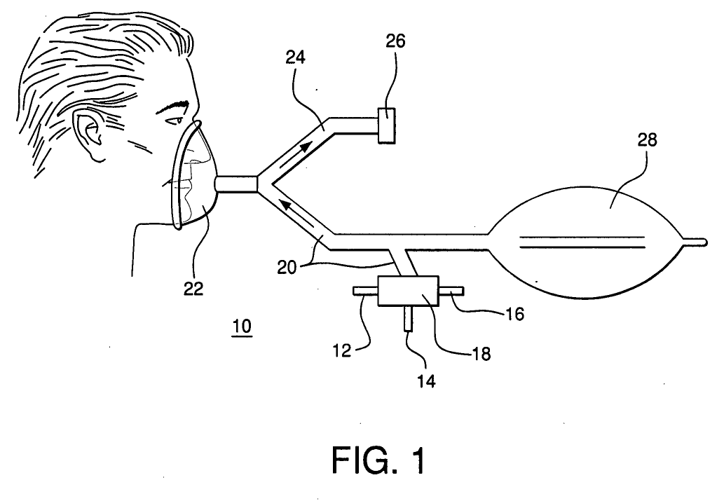

As shown in FIG. 1, iNO therapy can be performed by administering a gas mixture, which includes a therapeutically effective amount of iNO, to an individual using a portable apparatus 10. Apparatus 10 includes a dispenser 12 for oxygen gas, a dispenser 14 for nitric oxide gas, and an optional dispenser 16 for helium gas. As used herein, a “dispenser” is a canister, cylinder, container, or other like apparatus capable of storing and dispensing a gas. The dispensers can be attached to apparatus 10 as shown, or can be separate articles connected to apparatus by means of a hose or tube. The dispensers that are separate include fixed and semi-fixed dispensers such as the cylinders used in party supply stores to inflate balloons with helium.

Apparatus 10 further includes a gas blender 18, which mixes the gases that are released from dispensers 12, 14, 16 to create a gas mixture. Gas blender 18 preferably mixes the gases to create a substantially homogenous mixture of the gases. Because portability of apparatus 10 is a preferred feature, it is preferred that, if a power source is necessary to run gas blender 18, that battery power or other similar means be used. However, it is contemplated that gas blender 18 can function from other power sources.

Once the gases are mixed into a gas mixture by gas blender 18, an individual breathes from facemask 22 causing the gas mixture to travel through inspiratory limb 20. Face mask 22 can be a full face mask as is found with full face respirators used in emergency response settings, it can be a half face mask, or it can be any other mask that substantially covers at least a portion of an individual's face, predominately around the mouth and nose area. The material of face mask 22 is preferably a rubber or other similar material that provides a seal between the face mask and the skin of the individual using apparatus 10 such that the gas mixture does not leak or escape from apparatus 10. Alternatively, the material of face mask 22 can comprise a substantially rigid material with a rubber-like material around the perimeter of the face mask.

When the individual exhales, the expiration travels through expiratory limb 24 and exits through register 26. The exit of the expiration can create a back pressure in apparatus 10 that allows a continuous positive airway pressure to be achieved, which consequently can increase lung recruitment and improve treatment efficacy.

Apparatus 10 can include additional features. For example, as shown in FIG. 1, apparatus 10 can include an anesthesia bag 28. The anesthesia bag can act as an inspiratory reservoir, storing excess gas mixture that is dispensed from dispensers 12, 14, 16, but not inhaled by the individual.

Apparatus 10 can include a strap that fits around an individual's head such that face mask 22 is secured against the face of the individual. The strap can be made from an elastic material or, alternatively, the strap can be adjustable so as to allow apparatus 10 to be used with individuals with different head shapes and sizes.

Apparatus 10 can include instrumentation and measurement means that allow for the measurement of data such as flow rate of each of the gases, concentrations of each of the gases in the gas mixture, inspiration rate, expiration rate, availability of gas in each of the dispensers, pressure in the inspiratory limb, and so on.

Apparatus 10 can include control devices that control the amount of gas dispensed from each of dispensers 12, 14, 16. Apparatus 10 can also control other functions such as the amount of the gas mixture inhaled and the pressure of the gas mixture in the apparatus. The controls can be manually operated (e.g., manually operated valves) or automated (e.g., computer controlled blowers or fans).

Apparatus 10 is beneficial in that it can be stored at locations such as dive sites so that immediate treatment can be given to an individual suffering from DCS. It is contemplated that the apparatus can be stored in much the same way as diving masks and other similar equipment are stored. This immediate response to DCS can be the difference between an individual recovering from DCS and an individual not recovering from DCS.

Apparatus 10 also is beneficial because it does not provide a significant impediment to close medical observation and treatment. Unlike HBO therapy, medical personnel can closely monitor and treat an individual undergoing iNO therapy using apparatus 10. For example, medical personnel can suture a wound on the leg of an individual at the same time the individual is being treated for DCS using the apparatus as a means of iNO therapy.

It will be appreciated by those skilled in the art, that the present invention may be practiced in various alternate forms and configurations. The previously detailed description of the disclosed embodiments is presented for purposes of clarity of understanding only, and no unnecessary limitations should be implied therefrom.

What is claimed is:

1. A method for treating an individual with decompression sickness, the method comprising:
administering a gas mixture to lungs of the individual, the gas mixture comprising oxygen and a therapeutically effective amount of nitric oxide gas.

2. The method of claim 1 wherein the therapeutically effective amount of nitric oxide gas is from about 1 ppm to about 100 ppm in the gas mixture.

3. The method of claim 2 wherein the therapeutically effective amount of nitric oxide gas from about 5 ppm to about 80 ppm in the gas mixture.

4. The method of claim 1 wherein the gas mixture further comprises helium.

5. The method of claim 4 wherein the helium is from about 50% to about 80% of the gas mixture based on volume of the gas mixture.

6. The method of claim 1 wherein the gas mixture is administered under positive pressure.

7. The method of claim 1 wherein the gas mixture is administered at a pressure greater than about 1 atmosphere.

8. The method of claim 7 wherein the gas mixture is administered at a pressure greater than about 2 atmospheres.

9. A method for preventing decompression sickness, the method comprising administering a gas mixture to at least one lung of a person, the gas mixture comprising oxygen and a therapeutically effective amount of nitric oxide gas.

10. The method of claim 9 wherein the therapeutically effective amount of nitric oxide gas is from about 1 ppm to about 100 ppm in the gas mixture.

11. The method of claim 10 wherein the therapeutically effective amount of nitric oxide gas is from about 5 ppm to about 80 ppm in the gas mixture.

12. A therapeutic gas mixture comprising:
oxygen, helium, and a therapeutically effective amount of gaseous nitric oxide,
wherein the nitric oxide, oxygen, and helium are mixed to create the therapeutic gas mixture.

13. The gas mixture of claim 12 wherein the therapeutically effective amount of gaseous nitric oxide is from about 1 ppm to about 100 ppm in the gas mixture.

14. The gas mixture of claim 13 wherein the therapeutically effective amount of gaseous nitric oxide is from about 5 ppm to about 100 ppm in the gas mixture.

15. The gas mixture of claim 12 wherein the helium is from about 50% to about 80% of the gas mixture based on the volume of the gas mixture.

16. The gas mixture of claim 12 wherein the gas mixture is administered under positive air pressure.

17. The gas mixture of claim 12 wherein the gas mixture is administered to a person suffering from decompression sickness.

18. The gas mixture of claim 12 wherein the gas mixture is substantially homogeneous.

19. A device for administering a gas mixture to an individual, the device comprising:
at least one dispenser of oxygen gas;
at least one dispenser of helium gas;
at least one dispenser of nitric oxide gas;
a gas blender to mix the oxygen gas, the helium gas and the nitric oxide gas into a gas mixture;
an inspiratory passage;
a face mask substantially conformable with the individual's face; and
an expiratory passage;
wherein when the individual places the device on the individual's face and inhales, the gas mixture travels from the gas blender, through the inspiratory passage to the face mask where the gas mixture is inhaled by the individual, and when the individual exhales a breath, the breath travels from the face mask through the expiratory passage.

20. A device of claim 19 wherein the gas mixture is a substantially homogenous blend of the oxygen gas, the helium gas, and the nitric oxide gas.

21. The device of claim 19 wherein the device further comprises a register at an end of the expiratory passage opposite the individual, the register through which the breath of the individual exits, creating a back pressure, thereby providing continuous positive airway pressure.

22. The device of claim 21 wherein the device further comprises an apparatus to measure air pressure in the device.

23. The device of claim 19 wherein the face mask is substantially conformable to the individual's face.

24. The device of claim 23 wherein the face mask covers only a portion of the individual's face.

25. The device is claim 19 wherein the device further comprises an apparatus to monitor the amount of the gas mixture inhaled by the individual.

26. The device of claim 19 wherein the device further comprises an apparatus to monitor the amount of gaseous nitric oxide inhaled by the individual.

27. The device of claim 19 wherein the at least one dispenser of oxygen gas, the at least one dispenser of helium gas, and the at least one dispenser of nitric oxide gas are connected to the device via tubing.
